(19) United States
(12) Patent Application Publication
(10) Pub. No.: US 2025/0001651 A1
(43) Pub. Date: Jan. 2, 2025

(54) MOLD UNIT FOR MANUFACTURING MICROSTRUCTURES

(71) Applicant: IUCF-HYU (INDUSTRY-UNIVERSITY COOPERATION FOUNDATION HANYANG UNIVERSITY), Seoul (KR)

(72) Inventors: Yong Hee KIM, Seoul (KR); Shayan FAKHRAEILAHIJI, Seoul (KR)

(73) Assignee: IUCF-HYU (INDUSTRY-UNIVERSITY COOPERATION FOUNDATION HANYANG UNIVERSITY), Seoul (KR)

(21) Appl. No.: 18/708,703

(22) PCT Filed: Nov. 9, 2022
(86) PCT No.: PCT/KR2022/017518

§ 371 Date: May 9, 2024

(30) Foreign Application Priority Data
Nov. 10, 2021 (KR) 10-2021-0153551

(51) Int. Cl. B29C 39/26; B29L 31/00; B81C 1/00

(52) U.S. Cl. CPC B29C 39/26; B81C 1/00071; B29L 2031/7544; B29L 2031/756

(57) ABSTRACT

Disclosed is a mold unit for manufacturing microstructures, the mold unit comprising a first mold for manufacturing microstructures in which a plurality of microneedles are formed on one surface of a base layer. The first mold comprises: a first base part in which first needle grooves for forming the microneedles are formed on the upper surface, and which includes a first embankment protruding to a predetermined height around the region where the first needle grooves are formed; a first edge part which is provided along the periphery of the first base part at a predetermined distance from the first base part, and of which the upper end is provided higher than the first embankment; and a first extension part which extends from the first base part to the first edge part, and of which the upper surface is positioned lower than the region where the first needle grooves are formed.

### TECHNICAL FIELD

The present invention relates to a mold unit for manufacturing microstructures, and more specifically, to a mold unit for manufacturing microstructures capable of manufacturing the microstructures into a uniform size.

### BACKGROUND ART

Routes of administration for delivering drugs to the human body include oral administration, injection administration, and transdermal administration. The oral administration is convenient administration that may increase patient's compliance with the medication, in which active ingredients are delivered to the human body in the form of capsules, tablets, and syrups. However, the active ingredients may be inactivated due to first-pass metabolism in the liver, and in fact, the absorption rate of biopharmaceuticals is relatively low. Therefore, in order to express accurate and rapid medicinal effects such as drugs and therapeutic agents, the drugs are injected into the human body by penetrating the skin barrier through the injection administration. When the drugs are delivered through the injection administration, there is an advantage that the activity of active ingredients is maintained, but there are disadvantages such as the risk of infection, incorrect dose administration, phobias, and pain.

In order to overcome the limitations of oral and injection route administration, various microstructure transdermal drug delivery systems including minimally penetrating microneedles have been developed. Microstructures are mainly manufactured in biodegradable/dissolving, solid, coating, and hollow forms. The biodegradable microstructure is a transdermal delivery system in which various materials including polymers and active ingredients (API/cosmetics or pharmaceuticals) are formulated in the form of microneedles, and loaded materials are dissolved by body fluids after the microstructure is inserted into the skin so that the drugs may be delivered without pain.

A mold casting manufacturing method is used as a method for manufacturing microstructures. In the mold casting manufacturing method, a mold is filled with a composition using centrifugal force or vacuum and then dried.

FIG. 17 is a view showing various existing mold structures and microstructures manufactured therefrom.

Referring to FIG. 17, when a mold 51 having no wall surface is used, a base layer 71 of a microstructure 70 is widely manufactured, resulting in an additional process of removing unnecessary portions.

When molds 52 and 53 having a wall surface are used, a composition is stuck to a wall and an edge thickness thereof is larger than a middle thickness of the base layer 71. The edge thickness may cause a problem in penetration uniformity because each region has a different depth of penetration into the skin when the microstructure is applied to the skin. In addition, since it is difficult to adjust the thickness of the microstructure, when a composition having an amount greater than or equal to the loading amount is supplied, a problem occurs in that the thickness of the base layer 81 becomes large.

### DISCLOSURE

### Technical Problem

The present invention provides a mold unit for manufacturing microstructures capable of manufacturing a uniform thickness of a base layer.

In addition, the present invention provides a mold unit for quantitatively using a composition and recovering a residual composition.

In addition, the present invention provides a mold unit for manufacturing microstructures capable of manufacturing a plurality of microstructures through a single manufacturing process.

### Technical Solution

A mold unit for manufacturing microstructures according to the present invention includes a first mold for manufacturing microstructures in which a plurality of microneedles are formed on one surface of a base layer, in which the first mold includes: a first base part in which first needle grooves for forming the microneedles are formed on an upper surface of the first mold, and which includes a first embankment protruding with a predetermined height around a region in which the first needle grooves are formed; a first edge part which is provided along a periphery of the first base part while being spaced apart from the first base part at a predetermined distance, and in which an upper end of the first edge part is provided higher than the first embankment; and a first extension part which extends from the first base part to the first edge part, and in which an upper surface of the first extension part is positioned lower than the region in which the first needle grooves are formed.

In addition, the first extension unit may be formed with a flow path.

In addition, the mold unit may further include a storage container having an inner space with an open upper surface, in which a lower end of the first edge part is disposed on an upper end of the storage container, in which a bottom surface of the storage container may be spaced apart from a lower surface of the first base part at a predetermined distance.

In addition, the storage container may be separable from the first mold.

In addition, the bottom surface of the storage container may be formed with an opening, and the mold unit may further include a valve for opening/closing the opening.

In addition, one side surface of the embankment, which is adjacent to the region formed with the needle grooves, may be provided as an inclined surface.

In addition, the flow path may extend from the upper surface of the first extension part, and may be inclined downward toward a center of the first base part.

In addition, the mold unit may further include a second mold that is stackable with the first mold, in which the second mold may include: a second base part in which second needle grooves for forming the microneedles are formed on an upper surface of the second mold, and which includes a second embankment protruding with a predetermined height around a region in which the second needle grooves are formed; a second edge part which is provided along a periphery of the second base part while being spaced apart from the second base part at a predetermined distance, and in which an upper end of the first edge part is provided higher than the second embankment; and a second extension part which extends from the second base part to the second edge part, and in which an upper surface of the second extension part is positioned lower than the region in which the second needle grooves are formed.

In addition, the flow path may be formed toward the region in which the second needle grooves are formed in the second base part.

### Advantageous Effects

According to the present invention, a portion of the composition is supplied into the needle groove, the remaining portion thereof is filled up to the upper end of the embankment, and the residual composition is recovered by flowing over the embankment, so that the base layer may be manufactured with a uniform thickness.

In addition, according to the present invention, the residual composition flowing over the embankment is recovered from a space between the base part and the edge part, so that the residual composition may be reused.

In addition, according to the present invention, the recovered residual composition is supplied into another mold, so that a plurality of microstructures may be manufactured through a single manufacturing process.

### DESCRIPTION OF DRAWINGS

FIG. 1 is a perspective view showing a mold unit for manufacturing microstructures according to one embodiment of the present invention.

FIG. 2 is a plan view showing the mold unit for manufacturing microstructures of FIG. 1.

FIG. 3 is a sectional view showing the mold unit for manufacturing microstructures of FIG. 1.

FIGS. 4 and 5 are views sequentially showing a process of manufacturing microstructures using the mold unit for manufacturing microstructures of FIG. 1.

FIG. 6 is a view showing microstructures manufactured using the mold unit for manufacturing microstructures of FIG. 1.

FIG. 7 is a sectional view showing a portion of a mold unit for manufacturing microstructures according to another embodiment of the present invention.

FIG. 8 is a view showing the microstructures manufactured using the mold unit for manufacturing microstructures of FIG. 7.

FIG. 9 is a perspective view showing a mold unit for manufacturing microstructures according to still another embodiment of the present invention.

FIG. 10 is a plan view showing the mold unit for manufacturing microstructures of FIG. 9.

FIG. 11 is a sectional view showing the mold unit for manufacturing microstructures of FIG. 9.

FIG. 12 is a view sequentially showing a process of manufacturing microstructures using the mold unit for manufacturing microstructures of FIG. 9.

FIG. 13 is a sectional view showing a mold unit for manufacturing microstructures according to still another embodiment of the present invention.

FIG. 14 is a view sequentially showing a process of manufacturing microstructures using the mold unit for manufacturing microstructures of FIG. 13.

FIGS. 15 and 16 are perspective views showing mold units for manufacturing microstructures according to mutually different embodiments of the present invention.

FIG. 17 is a view showing various existing mold structures and microstructures manufactured therefrom.

### BEST MODE FOR INVENTION

A mold unit for manufacturing microstructures according to the present invention includes a first mold for manufacturing microstructures in which a plurality of microneedles are formed on one surface of a base layer, in which the first mold includes: a first base part in which first needle grooves for forming the microneedles are formed on an upper surface of the first mold, and which includes a first embankment protruding with a predetermined height around a region in which the first needle grooves are formed; a first edge part which is provided along a periphery of the first base part while being spaced apart from the first base part at a predetermined distance, and in which an upper end of the first edge part is provided higher than the first embankment; and a first extension part which extends from the first base part to the first edge part, and in which an upper surface of the first extension part is positioned lower than the region in which the first needle grooves are formed.

### MODE FOR INVENTION

Hereinafter, preferred embodiments of the present invention will be described in detail with reference to the accompanying drawings. However, the present invention may be embodied in different forms and should not be construed as limited to the embodiments set forth herein. Rather, the embodiments are provided so that this disclosure will be thorough and complete, and will fully convey the scope of the present invention to those skilled in the art.

In the present specification, it will be understood that when an element is referred to as being “on” another element, it can be formed directly on the other element or intervening elements may be present. In the drawings, the thicknesses of layers and regions are exaggerated for clarity.

In addition, it will be also understood that although the terms first, second, third, etc. may be used herein to describe various elements, these elements should not be limited by these terms. These terms are only used to distinguish one element from another element. Thus, a first element in some embodiments may be termed a second element in other embodiments without departing from the teachings of the present invention. Embodiments explained and illustrated herein include their complementary counterparts. As used herein, the term “and/or” includes any and all combinations of one or more of the associated listed elements.

The singular expression also includes the plural meaning as long as it does not differently mean in the context. In addition, the terms “comprise”, “have” etc., of the description are used to indicate that there are features, numbers, steps, elements, or combination thereof, and they should not exclude the possibilities of combination or addition of one or more features, numbers, operations, elements, or a combination thereof. Furthermore, it will be understood that when an element is referred to as being “connected” or “coupled” to another element, it may be directly connected or coupled to the other element or intervening elements may be present.

In addition, when detailed descriptions of related known functions or constitutions are considered to unnecessarily cloud the gist of the present invention in describing the present invention below, the detailed descriptions will not be included.

A mold unit for manufacturing microstructures according various embodiments described below may manufacture microstructures capable of delivering a drug to the human body. The microstructure has a structure in which a thin base layer is coupled to a plurality of microneedles formed on one surface of the base layer, and the needles are inserted into the skin tissue to deliver a drug. The microstructure is manufactured by filling needle grooves of the mold unit for manufacturing microstructures with a composition. The composition may be a biocompatible or biodegradable material. The biocompatible or biodegradable material is a material that is substantially non-toxic to the human body, chemically inactive, and non-immunogenic, and has the advantage of being dissolved after penetration into the human body.

The type of the biocompatible material is not particularly limited, and examples thereof may include hyaluronic acid, polyester, polyhydroxyalkanoate (PHAs), poly(α-hydroxyacid), poly(β-hydroxyacid), poly(3-hydroxybutyrate-co-valerate; PHBV), poly(3-hydroxyproprionate; PHP), poly(3-hydroxyhexanoate; PHH), poly(4-hydroxyacid), poly(4-hydroxybutyrate), poly(4-hydroxyvalerate), poly(4-hydroxyhexanoate), poly(esteramide), polycaprolactone, polylactide, polyglycolide, poly(lactide-co-glycolide; PLGA), polydioxanone, polyorthoester, polyetherester, polyanhydride, poly(glycolic acid-co-trimethylene carbonate), polyphosphoester, polyphosphoester urethane, poly(amino acid), polycyanoacrylate, poly(trimethylene carbonate), poly(iminocarbonate), poly(tyrosine carbonate), polycarbonate, poly(tyrosine arylate), polyalkylene oxalate, polyphosphazenes, PHA-PEG, ethylene vinyl alcohol copolymer (EVOH), polyurethane, silicone, polyester, polyolefin, polyisobutylene, ethylene-alphaolefin copolymer, styrene-isobutylene-styrene triblock copolymer, acrylic polymer and copolymer, vinyl halide polymer and copolymer, polyvinyl chloride, polyvinyl ether, polyvinyl methyl ether, polyvinylidene halide, polyvinylidene fluoride polyvinylidene chloride, polyfluoroalkene, polyperfluoroalkene, polyacrylonitrile, polyvinyl ketone, polyvinyl aromatics, polystyrene, polyvinyl ester, polyvinyl acetate, ethylene-methyl methacrylate copolymer, acrylonitrile-styrene copolymer, ABS resin and ethylene-vinyl acetate copolymer, polyamide, alkyd resin, polyoxymethylene, polyimide, polyether, polyacrylate, polymethyl methacrylate, polyacrylic acid-co-maleic acid, chitosan, dextran, cellulose, heparin, alginate, inulin, starch, or glycogen, and the examples thereof may include at least one selected from the group consisting of hyaluronic acid, polyester, polyhydroxyalkanoate (PHAs), poly(α-hydroxyacid), poly(β-hydroxyacid), poly(3-hydroxybutyrate-co-valerate; PHBV), poly(3-hydroxyproprionate; PHP), poly(3-hydroxyhexanoate; PHH), poly(4-hydroxyacid), poly(4-hydroxybutyrate), poly(4-hydroxyvalerate), poly(4-hydroxyhexanoate), poly(esteramide), polycaprolactone, polylactide, polyglycolide, poly(lactide-co-glycolide; PLGA), polydioxanone, polyorthoester, polyetherester, polyanhydride, poly(glycolic acid-co-trimethylene carbonate), polyphosphoester, polyphosphoester urethane, poly(amino acid), polycyanoacrylate, poly(trimethylene carbonate), poly(iminocarbonate), poly(tyrosine carbonate), polycarbonate, poly(tyrosine arylate), polyalkylene oxalate, polyphosphazenes, PHAPEG, chitosan, dextran, cellulose, heparin, alginate, inulin, starch, and glycogen.

When the microstructure is a solid-type microneedle loaded with a biocompatible or biodegradable material, a drug may be additionally loaded. The drug refers to a broad concept, and includes not only therapeutic agents for therapeutic purposes of consultation, but also energy, nanocomponents, cosmetic components (for example, anti-wrinkle agents, skin aging inhibitors, and skin whitening agents), cell culture media, and the like.

Specifically, the therapeutic agent includes a chemical drug, a protein/peptide drug, a peptide drug, a nucleic acid molecule for gene therapy, and the like.

For example, the therapeutic agent may include anti-inflammatory agents, analgesics, anti-arthritic agents, antispasms, antidepressants, antipsychotic drugs, tranquilizers, anxiolytics, drug antagonists, anti-Parkinson's disease drugs, cholinergic agonists, anticancer agents, anti-angiogenic agents, immunosuppressive agents, antiviral agents, antibiotics, appetite suppressants, analgesics, anticholinergic agents, antihistamines, anti-migraine agents, hormonal agents, coronary vasodilator, cerebrovascular or peripheral vascular dilator, contraceptives, antithrombotic agents, diuretics, antihypertensives, cardiovascular disease therapeutic agents, and the like.

In particular, the protein/peptide drug may include hormones, hormone analogs, enzymes, enzyme inhibitors, signal transduction proteins or portions thereof, antibodies or portions thereof, short-chain antibodies, binding proteins or binding domains thereof, antigens, adhesion proteins, structural proteins, regulatory proteins, toxic proteins, cytokines, transcriptional regulatory factors, blood coagulation factors, vaccines, and the like. More specifically, the protein/peptide drug may include insulin, insulin-like growth factor 1 (IGF-1), growth hormone, erythropoietin, granulocyte-colony stimulating factors (G-CSFs), granulocyte/macrophagecolony stimulating factors (GM-CSFs), interferon alpha, interferon beta, interferon gamma, interleukin-1 alpha and beta, interleukin-3, interleukin-4, interleukin-6, interleukin-2, epidermal growth factors (EGFs), calcitonin, adrenocorticotropic hormone (ACTH), tumor necrosis factor (TNF), atobisban, buserelin, cetrorex, deslorelin, desmopressin, dinorphin A (1-13), elcatonin, eleidosin, eptifibatide, growth hormone releasing hormone-II (GHRHII), gonadorelin, goserelin, histrelin, leuprorelin, lypressin, octreotide, oxytocin, pitressin, secretin, syncalide, teripressin, thymopentin, thymosin al, triptorelin, bivalirudin, carbetocin, cyclosporine, exedine, lanreotide, luteinizing hormone-releasing hormone (LHRH), nafarelin, parathyroid hormone, pramlintide, enfuvirtide (T-20), thymalfasin, and ziconotide.

The mold unit for manufacturing microstructures may have various shapes. The drawings of the present invention show that the mold unit has a rectangular shape, but the present invention is not limited thereto, and may have a circular or polygonal shape. According to such a shape, base layers having rectangular, circular, and polygonal shapes may be manufactured.

Hereinafter, a mold unit for manufacturing microstructures according to the present invention will be described in detail.

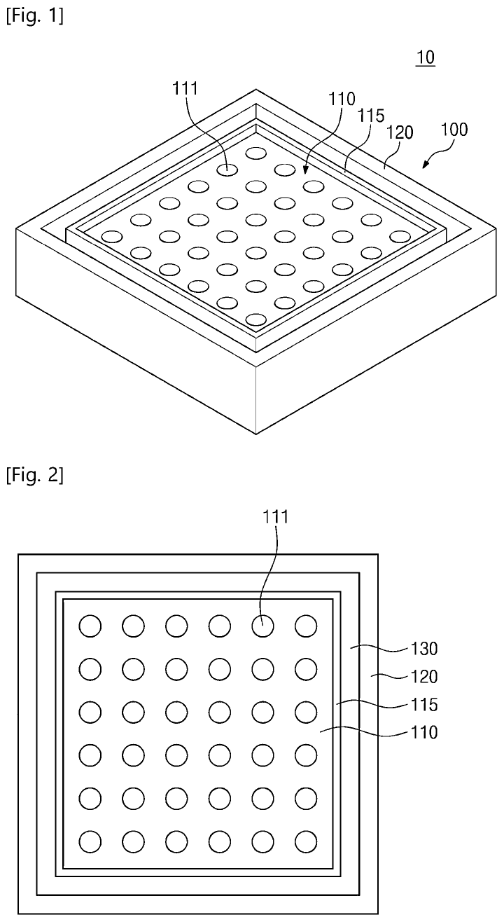

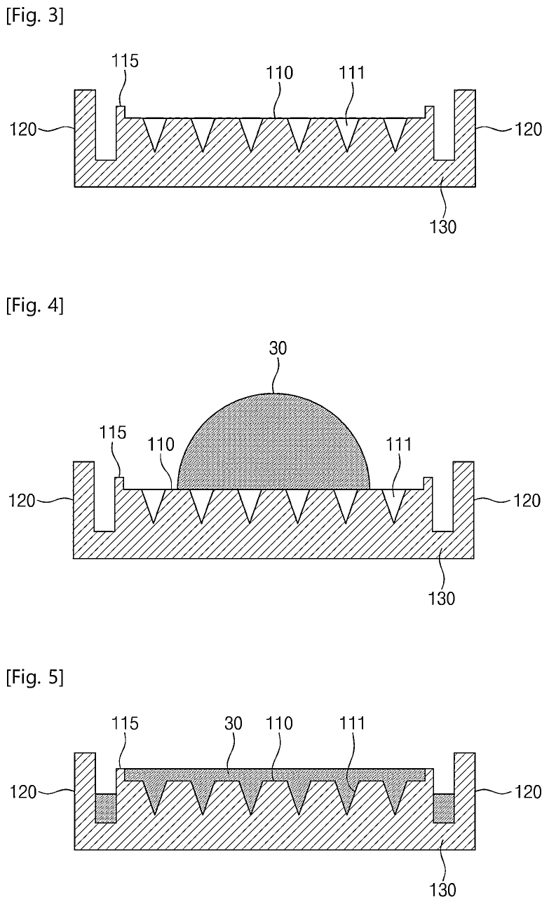

FIG. 1 is a perspective view showing a mold unit for manufacturing microstructures according to one embodiment of the present invention, FIG. 2 is a plan view showing the mold unit for manufacturing microstructures of FIG. 1, and FIG. 3 is a sectional view showing the mold unit for manufacturing microstructures of FIG. 1.

Referring to FIG. 1, a mold unit 10 for manufacturing microstructures includes a first mold 100.

The first mold 100 is provided for manufacturing one microstructure. The first mold 100 includes a first base part 110, a first edge part 120, and a first extension part 130.

The first base part 110 is a rectangular plate having predetermined width and thickness, and has first needle grooves 111 formed on an upper surface thereof. The first needle grooves 111 are formed with a predetermined depth in a predetermined number and arrangement.

The upper surface of the first base part 110 is formed with a first embankment 115 along a periphery of a region in which the first needle grooves 111 are formed. The first embankment 115 is formed in a rectangular shape and protrudes with a predetermined height. An upper end of the first embankment 115 is positioned higher than the upper surface of the region in which the first needle grooves 111 are formed. One side surface of the first embankment 115, which is adjacent to the region in which the first needle grooves 111 are formed, is provided perpendicular to the region in which the first needle grooves 111 are formed.

The first edge part 120 is spaced apart from the first base part 110, and is provided along a periphery of the first base part 110. An interval between first edge part 120 and the first base part 110 is larger than a thickness of the first embankment 115. The first edge part 120 is formed in a rectangular shape, in which an upper end of the first edge part 120 is positioned higher than the upper end of the first embankment 115.

The first extension part 130 extends from the first edge part 120 to the first base part 110. An upper surface of the first extension part 130 is positioned lower than the upper surface of the region in which the first needle grooves 111 are formed.

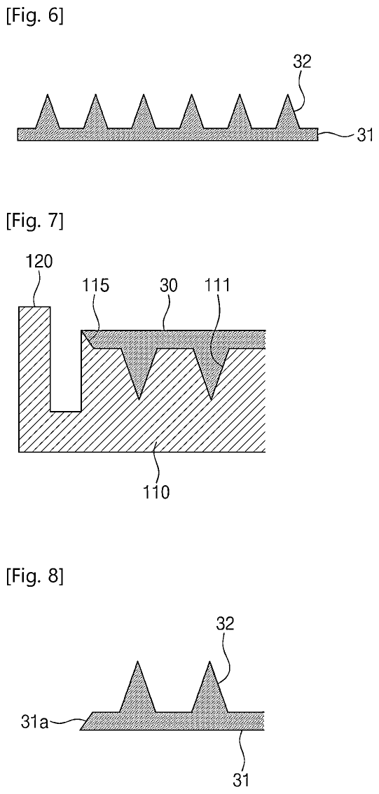

FIGS. 4 and 5 are views sequentially showing a process of manufacturing microstructures using the mold unit for manufacturing microstructures of FIG. 1, and FIG. 6 a view showing microstructures manufactured using the mold unit for manufacturing microstructures of FIG. 1.

Referring to FIGS. 4 to 6, a composition 30 is supplied to the upper surface of the first base part 110. The composition 30 is spread along the upper surface of the first base part 110 by centrifugal force, air pressure, or vacuum pressure, in which a portion thereof is injected into the first needle grooves 111, and the remaining portion thereof is filled up to the upper end of the first embankment 115. In addition, the residual composition 30 is introduced into a space between the first base part 110 and the first edge part 120 by flowing over the first embankment 115, and is recovered.

The composition 30 injected into first needle grooves 111 forms a microneedle 32, and the composition 30 confined in the first embankment 115 forms a base layer 31. An end portion of the base layer 31 is formed perpendicular to a side surface of the first embankment 115.

According to the manufacturing method described above, the base layer 31 has the same thickness as a height of the first embankment 115 even when the composition 30 is supplied in a fixed quantity or in excess. Therefore, it is possible to manufacture microstructures 31 and 32 having a uniform size. In addition, the excessively supplied composition 30 is recovered in the space between the first base part 110 and the first edge part 120, and thus may be reused.

FIG. 7 is a sectional view showing a portion of a mold unit for manufacturing microstructures according to another embodiment of the present invention, and FIG. 8 is a view showing the microstructures manufactured using the mold unit for manufacturing microstructures of FIG. 7.

Referring to FIGS. 7 and 8, one side surface of the first embankment 115, which is adjacent to the region in which the first needle grooves 111 are formed, is provided as an inclined surface. The inclined surface forms an angle greater than 90° with the region in which the first needle grooves 111 are formed. According to the shape of the first embankment 115 described above, the formed end portion of the base layer 31 has an inclined surface 31a. The shape of the first embankment 115 may allow easy separation of the microstructures 31 and 32 from the first base part 110.

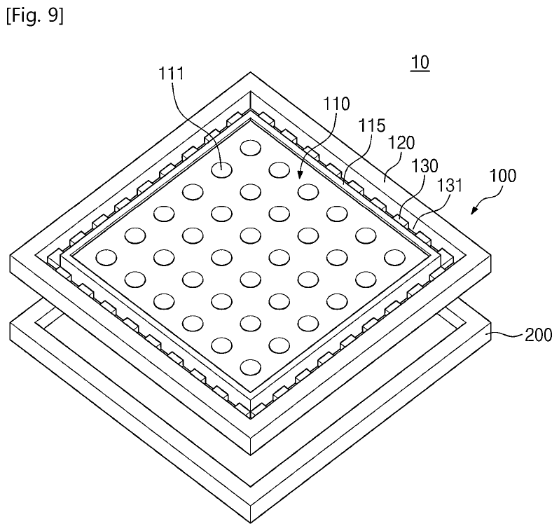

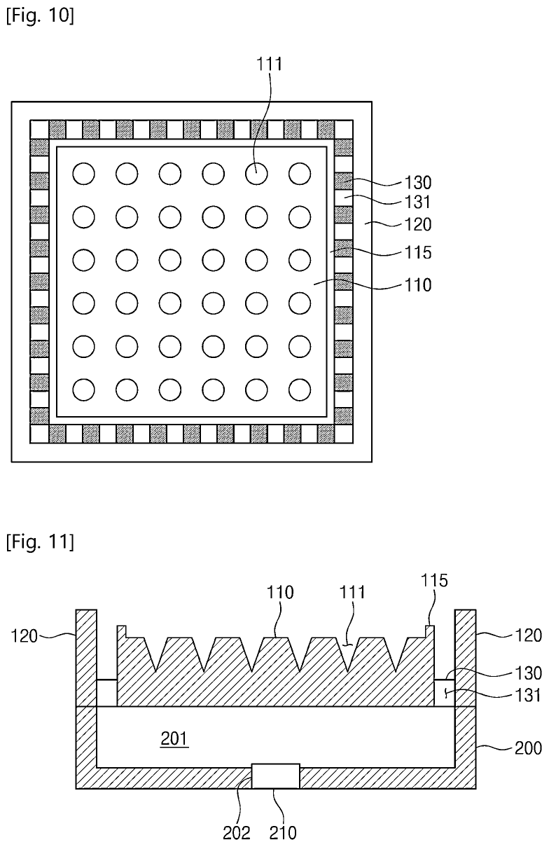

FIG. 9 is a perspective view showing a mold unit for manufacturing microstructures to still another embodiment of the present invention, FIG. 10 is a plan view showing the mold unit for manufacturing microstructures of FIG. 9, and FIG. 11 is a sectional view showing the mold unit for manufacturing microstructures of FIG. 9.

Referring to FIGS. 9 to 11, the first extension part 130 of the first mold 100 is formed with a flow path 131. The flow path 131 extends from the upper surface of the first extension part 130 to a lower surface. A plurality of flow paths 131 may be formed along a periphery of the first extension part 130 while being spaced apart from each other. According to one embodiment, the plurality of flow paths 131 may be formed at equal intervals along each side surface of the first base part 110. According to another embodiment, the plurality of flow paths 131 may be formed at a length corresponding to each side surface of the first base part 110.

The composition 30, which is introduced into the space between the first base part 110 and the first edge part 120, passes through the flow path and flows below the first extension part 130.

The mold unit 10 for manufacturing microstructures further includes a storage container 200.

The storage container 200 may have a size corresponding to the first mold 100, and has an inner space 201 having an open upper surface. The inner space 201 has a predetermined depth. The storage container 200 is positioned under the first mold 100, in which a lower end of the first edge part 120 is disposed on an upper end of the storage container 200. An inner bottom surface of the storage container 200 is spaced apart from the lower surface of the first base part 110 at a predetermined distance. The storage container 200 may be integrally coupled with the first mold 100. Alternatively, the storage container 200 may be separated from the first mold 100. The composition 30 passing through the flow path 131 is recovered in the storage container 200. The bottom surface of the storage container 200 is formed with an opening 202. The opening 202 is openable/closeable by a valve 210. According to one embodiment, the valve 210 slidably opens/closes the opening 201. According to another embodiment, the valve 210 may be provided as a cap that is separable from the storage container 200. According to still another embodiment, the valve 210 may control the degree of opening of the opening 201 in a rotating manner or a push-up manner.

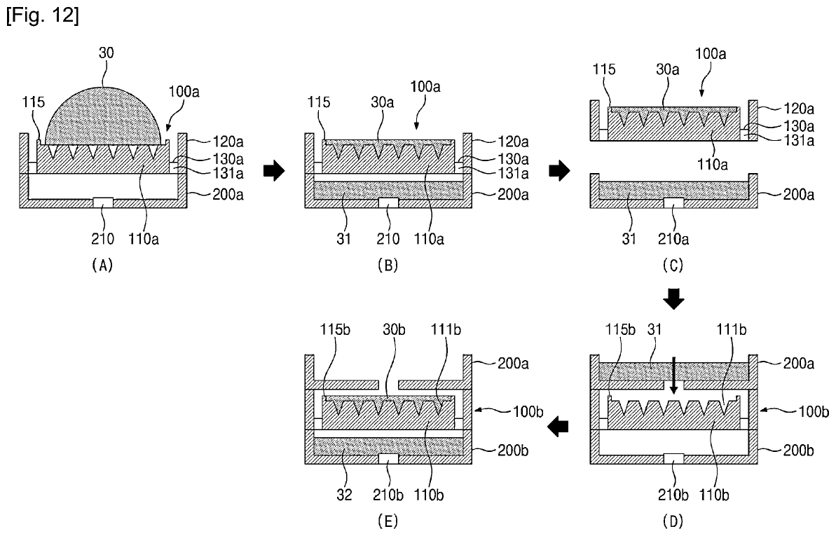

FIG. 12 is a view sequentially showing a process of manufacturing microstructures using the mold unit for manufacturing microstructures of FIG. 9.

Referring to FIG. 12, a composition 30 is supplied to an upper surface of the first base part 110a in a state in which one first mold 100a is disposed on an upper end of a storage container 200a. The composition 30 is spread along the upper surface of the first base part 110a, in which a portion thereof is injected into first needle grooves 111a, and the remaining portion thereof is filled up to the upper end of a first embankment 115a. In addition, a residual composition 31 is introduced into a space between the first base part 110a and a first edge part 120a by flowing over the first embankment 115a, and is recovered in the storage container 200a by passing through a flow path 131a.

When the composition 31 is recovered in the storage container 200a, the first mold 100a is separated from the storage container 200a, and the storage container 200a is mounted on an upper end of another first mold 100b. In addition, when a valve 210a is opened, the composition 31 recovered in the storage container 200a is supplied to a first base part 110b of another first mold 100b. The composition 31 is filled in a first dome part 115b of the first base part residual composition 32 is recovered in another storage container 200b.

By repeating the above-described process, a plurality of microstructures 30a and 30b may be manufactured. Since the composition 30 is used for only the quantity capable of filling the first embankments 115a and 115b and the remainder thereof is recovered and used for manufacturing another microstructure 30b, the microstructures 30a and 30b of a uniform size may be manufactured even though the composition 30 is not quantitatively supplied.

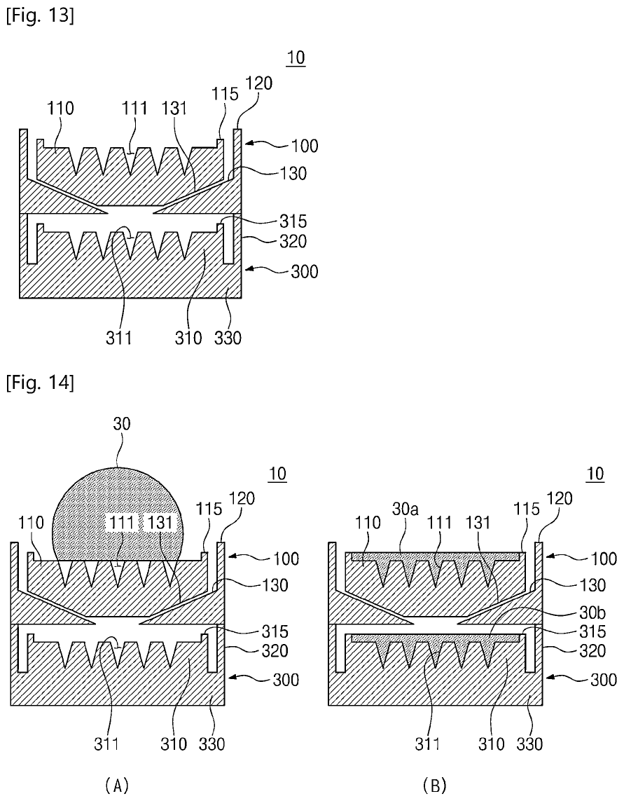

FIG. 13 is a sectional view showing a mold unit for manufacturing microstructures according to still another embodiment of the present invention.

Referring to FIG. 13, a mold unit 10 for manufacturing microstructures includes a first mold 100 and a second mold 300.

The first mold 100 has the same structure as the first mold described in FIG. 1, detailed descriptions thereof will be omitted.

A first extension part 130 is formed with a flow path 131. The flow path 131 is inclined downward from an upper surface of the first extension part 130. Specifically, the flow path 131 is inclined downward toward a center of the first base part 110.

The second mold 300 has the same width as the first mold 100, and is positioned under the first mold 100. The first mold 100 is disposed on an upper end of the second mold 300. The second mold 300 includes a second base part 310, a second edge part 320, and a second extension part 330.

The second base part 310 is a rectangular plate having the same width as the first base part 110, and has second needle grooves 311 formed on an upper surface thereof. The second needle grooves 311 are formed with a predetermined depth in a predetermined number and arrangement.

The upper surface of the second base part 310 is formed with a second embankment 315 along a periphery of a region in which the second needle grooves 311 are formed. The second embankment 315 is formed in a rectangular shape and protrudes with a predetermined height. An upper end of the second embankment 315 is positioned higher than the upper surface of the region in which the second needle grooves 311 are formed. The second embankment 315 has the same height as the first embankment 115. One side surface of the second embankment 315, which is adjacent to the region in which the second needle grooves 311 are formed, is provided perpendicular to the region in which the second needle grooves 311 are formed. Alternatively, one side surface of the second embankment 315 may be provided as an inclined surface.

The second edge part 320 is spaced apart from the second base part 310, and is provided along a periphery of the second base part 310. An interval between second edge part 320 and the second base part 310 is larger than a thickness of the second embankment 315. The second edge part 320 is formed in a rectangular shape, in which an upper end of the second edge part 320 is positioned higher than the upper end of the second embankment 315. A lower end of the first edge part 120 is disposed on the upper end of the second edge part 320.

The second extension part 330 extends from the second edge part 320 to the second base part 310. An upper surface of the second extension part 330 is positioned lower than the upper surface of the region in which the second needle grooves 311 are formed.

FIG. 14 is a view sequentially showing a process of manufacturing microstructures using the mold unit for manufacturing microstructures of FIG. 13.

Referring to FIG. 14, the composition 30 is supplied to the upper surface of the first base part 110 of the first mold 100 in a state in which the first mold 100 is disposed on the second mold 300. The composition 30 is spread along the upper surface of the first base part 110, in which a portion thereof is injected into the first needle grooves 111, and the remaining portion thereof is filled up to the upper end of the first embankment 115. In addition, the residual composition 30 is introduced into the space between the first base part 110 and the first edge part 120 by flowing over the first embankment 115, and is supplied to the upper surface of the second base part 310 by passing through the flow path 131. The composition 30 is spread along the upper surface of the second base part 310, in which a portion thereof is injected into the second needle grooves 311, and the remaining portion thereof is filled up to the upper end of the second embankment 315. In addition, the residual composition 30 is recovered into a space between the second base part 310 and the second edge part 320 by flowing over the second embankment 315.

Through the above-described manufacturing process, two microstructures 30a and 30b may be manufactured through a single manufacturing process. The number of stacked layers of the first mold 100 may be variously changed, and a plurality of microstructures 30a and 30b may be manufactured through a single manufacturing process according to the number of stacked layers of the first mold 100.

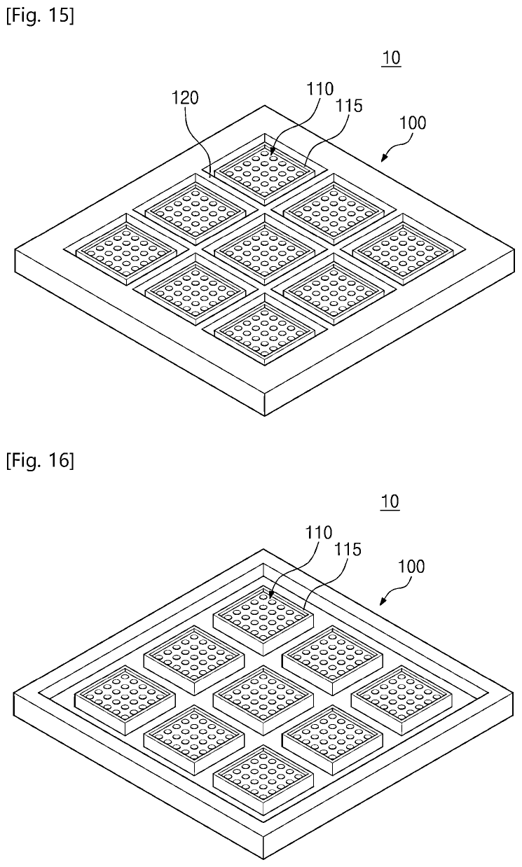

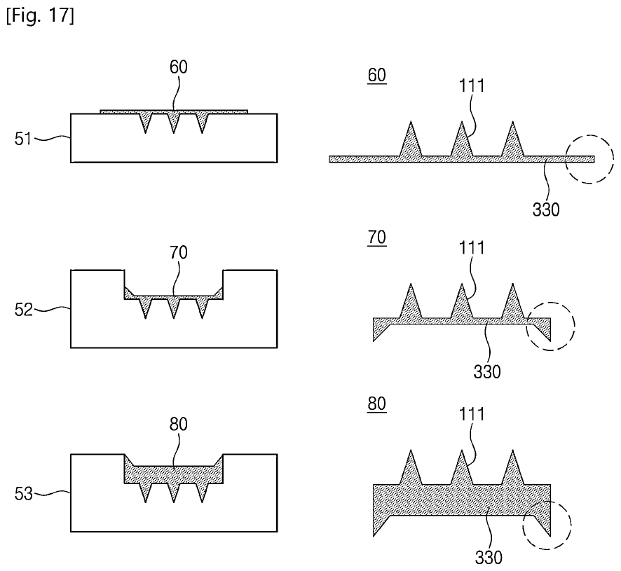

FIGS. 15 and 16 are perspective views showing mold units for manufacturing microstructures according to mutually different embodiments of the present invention.

Referring to FIG. 15, the first mold 100 may be provided with a plurality of first base part 110. The first base parts 110 may be arranged at predetermined intervals. In addition, the first edge part 120 may be provided in a region between the first base parts 110. The composition is supplied to each of the first base parts 110, and the residual composition flowing over the first embankment 115 is recovered into each recovery space, that is, the space between the first base part 110 and the first edge part 120.

Referring to FIG. 16, the plurality of first base parts 110 are arranged in the first mold 100. Unlike the FIG. 15, the first edge part is not provided between the first base parts 110. Therefore, in the composition supplied to each base part 110, a remaining amount of the composition flowing over the first embankment 115 may be recovered into one space.

While the present invention has been described in connection with the embodiments, it is not to be limited thereto but will be defined by the appended claims. In addition, it is to be understood that those skilled in the art can substitute, change or modify the embodiments in various forms without departing from the scope and spirit of the present invention.

### INDUSTRIAL APPLICABILITY

The mold unit for manufacturing microstructures according to the embodiment of the present invention may be used for manufacturing the microstructures capable of delivering a drug to the human body.

1. A mold unit for manufacturing microstructures, the mold unit comprising a first mold for manufacturing microstructures in which a plurality of microneedles are formed on one surface of a base layer,
wherein the first mold includes:
a first base part in which first needle grooves for forming the microneedles are formed on an upper surface of the first mold, and which includes a first embankment protruding with a predetermined height around a region in which the first needle grooves are formed;
a first edge part which is provided along a periphery of the first base part while being spaced apart from the first base part at a predetermined distance, and in which an upper end of the first edge part is provided higher than the first embankment; and
a first extension part which extends from the first base part to the first edge part, and in which an upper surface of the first extension part is positioned lower than the region in which the first needle grooves are formed.

2. The mold unit of claim 1, wherein the first extension part is formed with a flow path.

3. The mold unit of claim 2, further comprising a storage container having an inner space with an open upper surface, in which a lower end of the first edge part is disposed on an upper end of the storage container,
wherein a bottom surface of the storage container is spaced apart from a lower surface of the first base part at a predetermined distance.

4. The mold unit of claim 3, wherein the storage container is separable from the first mold.

5. The mold unit of claim 3, wherein the bottom surface of the storage container is formed with an opening, and
the mold unit further comprises a valve for opening/closing the opening.

6. The mold unit of claim 1, wherein one side surface of the embankment, which is adjacent to the region formed with the needle grooves, is provided as an inclined surface.

7. The mold unit of claim 2,
wherein the flow path extends from the upper surface of the first extension part, and is inclined downward toward a center of the first base part.

8. The mold unit of claim 7, further comprising a second mold that is stackable with the first mold,
wherein the second mold includes:
a second base part in which second needle grooves for forming the microneedles are formed on an upper surface of the second mold, and which includes a second embankment protruding with a predetermined height around a region in which the second needle grooves are formed;
a second edge part which is provided along a periphery of the second base part while being spaced apart from the second base part at a predetermined distance, and in which an upper end of the first edge part is provided higher than the second embankment; and
a second extension part which extends from the second base part to the second edge part, and in which an upper surface of the second extension part is positioned lower than the region in which the second needle grooves are formed.

9. The mold unit of claim 8, wherein the flow path is formed toward the region in which the second needle grooves are formed in the second base part.
